(19) United States
(12) Patent Application Publication
(10) Pub. No.: US 2005/0175508 A1
(43) Pub. Date: Aug. 11, 2005

(54) Particle counter for liquids

(76) Inventor: Barrington James Hill, Ledbury (GB)

Correspondence Address: IRA S. DORAN;Suite 200, 330 Roberts Street, East Hartford, CT, 06108, US

(21) Appl. No.: 10/923,927

(22) Filed: Aug. 23, 2004

(30) Foreign Application Priority Data
Feb. 11, 2004 (GB) 0402957.5

(51) Int. Cl. G01N 33/00

(52) U.S. Cl. 422/73

(57) ABSTRACT

A liquid particle counter includes an in situ light obscuration sensor (3). The sensor includes a sample cell (1) in the form of a glass tube of substantially circular cross-section for the passage of liquid, a light source (5) for directing a beam of light through the sample cell, and a photo-detector (7) for providing a signal representative of the amount of light passing through the sample cell (1). The signal is processed so as to produce an output (17) representative of a particle count.

### BACKGROUND TO THE INVENTION

The detection, sizing and counting of particles in liquids, such as water, has been known for many years. The primary reason for counting particles in liquids is to be able to determine and control the levels of contamination in the liquid. For example, in the context of drinking water, particle counting is used to monitor the cleanliness of drinking water, with the presence of particles being taken to indicate the possible presence of bacteria and similar organisms.

Particle counting has the benefit of providing very rapid results, but nevertheless cannot identify the nature of any particular particle. For example, a particle could be of an inert material or it could be biologically active. For safety, any particle is therefore considered to be biologically active.

It takes several days to monitor drinking water for bacteria such as by microscopic examination of filtered samples incubated on agar plates. By this time it is too late to prevent the supply of contaminated water, although it is possible to identify specific contaminants.

One application where particle counters could be useful lies in the monitoring of air conditioning systems for legionella bacteria. There is an acknowledged problem with air conditioning systems which use cooling water that may be aerosolised in that such systems may become sources of legionella. Such air conditioning systems are in effect small water treatment installations and consequently the use of particle counters could be effective to monitor the level of particulate contamination which may be representative of the level of legionella bacteria. If legionella bacteria can be monitored in this way, the air conditioning system can be serviced as and when necessary to control the presence of legionella.

### DESCRIPTION OF PRIOR ART

The problem is that known liquid particle counters are complex and costly and are therefore not considered to be sufficiently cost-effective for water treatment installations and air conditioning systems.

### OBJECT OF THE INVENTION

It is therefore an object of the present invention to provide a liquid particle counter which is sufficiently cost-effective to facilitate use, for example, in water treatment and air conditioning systems.

### SUMMARY OF THE INVENTION

According to the present invention there is provided a liquid particle counter comprising:
an in situ light obscuration sensor including a sample cell for the passage of liquid, a light source for directing a beam of light through the sample cell, and a photo-detector for providing a signal representative of the amount of light passing through the sample cell, the sample cell comprising a glass tube of substantially circular cross-section; and
means for processing the signal so as to produce an output representative of a particle count.

The use of a glass tube facilitates the use of push on fittings for the sample cell for the supply and removal of liquid.

The sensor may comprise a unitary sensor body. Such a sensor body enables other components, such as the light source and the photo-detector to be push fitted to the sensor body. The sensor body may be made of plastics material.

The light source may comprise a laser. The photo-detector may comprise a photo-transistor.

The sensor may include an aperture between the light source and the sample cell and an aperture between the sample cell and the photo-detector to define an illuminated viewing volume passing through the sample cell. The apertures are preferably substantially circular. The viewing volume may pass diametrically through the sample cell. Thus, where it strikes the outer surface of the sample cell, the beam of light may be substantially perpendicular to the outer surface.

The signal processing means may comprise a pulse height analyser. The pulse height analyser may determine size/count distribution. Alternatively, the signal processing means may comprise a comparator. The comparator may provide a number of pulses proportional to the number of particles detected in a predetermined time, such as one minute. Counting means may be provided to count the number of pulses and to provide a signal representative of the result.

### BRIEF DESCRIPTION OF THE DRAWING

For a better understanding of the present invention and to show more clearly how it may be carried into effect reference will now be made, by way of example, to the accompanying drawing which shows a diagrammatic cross-sectional view of one embodiment of a liquid particle counter according to the present invention.

### DESCRIPTION OF PREFERRED EMBODIMENT

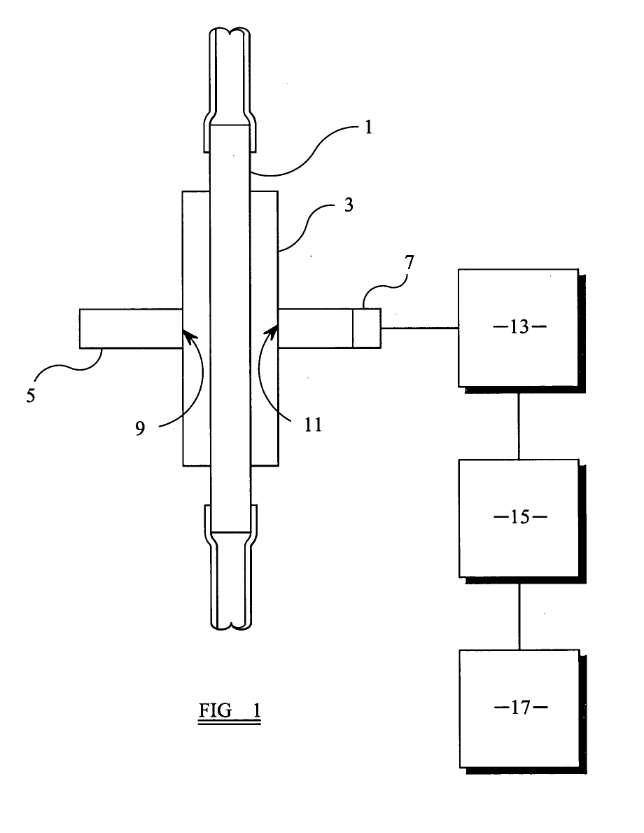

The liquid particle counter is shown in FIG. 1 in diagrammatic form and comprises a sample cell 1 which is made of conventional glass capillary tubing of substantially circular cross-section. In liquid counters it is standard practice to use a rectangular sample cell which is much more costly than glass tubing. It has generally been assumed that the sample cell must have planar faces which are perpendicular to the axis of a beam of light which, in use, passes through the sample cell. Surprisingly, we have found that when a small aperture is used the cylindrical surface of the sample cell appears to be substantially flat and perpendicular to the axis of the beam of light. The use of a cylindrical sample cell also offers the advantage over rectangular sample cells that push fit connections can be made thereby eliminating the need for costly connectors which are associated with connecting a rectangular sample cell to conventional cylindrical tubing. The glass tubing may have an outer diameter of up to about 7 mm, although the outer diameter may be any convenient size of about 5 mm or more. The internal diameter may be 1 mm or more. The smaller the internal diameter the higher the concentration limit (maximum number of particles per millilitre of liquid) that can be measured, but the larger the internal diameter the lower the pressure required to drive the liquid through the cell 1.

The cylindrical sample cell 1 is arranged within a sensor body, generally designated 3, the sensor body being machined or otherwise formed from a single piece of plastics or other suitable material. The use of a plastics material has the advantage of eliminating or reducing thermal effects which can give rise to problems with sensor bodies made of more conventional materials, such as aluminium. Further, the use of a plastics material allows other components of the particle counter to be a simple push fit into the plastics body. Thus, a light source 5 and a photo-detector 7 and also the sample cell 1 can be pushed together to form a sensor for the particle counter. A further unexpected advantage of making the sensor body 3 of a single block of plastics material is that condensation on the optical surfaces is substantially eliminated because the plastics material is less prone to condensation and because the components are sealed from the air. Such condensation can be a major problem for conventional liquid particle counters.

The light source 5, such as a laser or other source of suitably bright light, produces a beam of light which passes through an aperture 9 and substantially diametrically across the sample cell 1. Thereafter, the beam of light passes through a further aperture 11 and impinges upon the photo-detector 7. Thus the beam of light is substantially perpendicular to the cylindrical surfaces of the sample cell 1.

The apertures 9 and 11 are suitably circular and have a diameter of about 0.3 mm. The apertures together form a cylindrical viewing volume through the sample cell 1. It should be noted that the diameter of the cylindrical viewing volume is significantly less that the internal diameter of the sample cell 1 with the result that only part of the liquid sample passing through the sample cell is monitored. Such a sensor is known as an in situ sensor.

Because the diameter of the cylindrical viewing volume (about 0.3 mm) is small compared with the outer diameter of the tube forming the sample cell (about 7 mm), the incident light beam is for all practical purposes perpendicular to a planar face. It will be noted that the internal surface of the sample cell 1 is a glass/liquid interface and this interface is for practical purposes invisible.

Particle counters conventionally use photo-diodes as the photo-detector 7 in order to provide a high-speed signal. However, we have found that for liquid particle counters a photo-transistor can be used for the photo-detector in place of a photo-diode. A photo-transistor has the advantage that it is generally more sensitive and gives rise to lower signal noise. As a result the electronic circuit processing the output from the photo-transistor can be relatively simple, and therefore inexpensive. It should be noted that it is the recognition that a photo-transistor can be used in place of a photo-diode that is important. The implementation of a suitable electronic circuit is a straightforward task requiring no inventive skill.

In use of the particle counter, liquid passes at a substantially constant rate through the cylindrical sample cell 1 and as a particle passes through the viewing volume it blocks, or obscures, some of the light transmitted from the light source 5 to the photo-detector 7. The reduction in light reaching the photo-detector 7 results in a fluctuation in the current passing through the photo-detector which is approximately proportional to the square of the radius of the particle (assuming the particle to be substantially spherical).

The fluctuations in current are amplified in a processing circuit 13 to amplify and filter the signal and the resulting pulses are analysed by an analysing circuit 15. Conventionally, the analysing circuit 15 comprises a pulse height analyser (PHA) which outputs a particle size/count distribution to a suitable output device 17, such as a display, an alarm or computer-readable data. However, for the present purposes of detecting particulate contamination that includes bacteria or the like in a liquid such as water, it is only necessary to count the number of particles having a diameter greater than about 2 microns. This can readily be achieved, for example, by using a simple comparator to count the number of pulses exceeding a predetermined threshold in a predetermined time, such as one minute, and providing a single signal representative of the result, such as a signal in the range from 4 to 20 mA, or 0 to 5 volts or even as digital data. If the signal exceeds a predetermined threshold a signal can then be sent to the output device 17 to indicate that the water treatment installation or air conditioning system requires servicing.

Thus the liquid particle counter according to the present invention provides an inexpensive and effective means by which water quality in a water treatment installation or an air conditioning installation can be monitored for particulate contamination that includes bacteria such as legionella.

1. A liquid particle counter comprising:
an in situ light obscuration sensor including a sample cell for the passage of liquid, a light source for directing a beam of light through the sample cell, and a photo-detector for providing a signal representative of the amount of light passing through the sample cell, the sample cell comprising a glass tube of substantially circular cross-section; and
means for processing the signal so as to produce an output representative of a particle count.

2. The apparatus of claim 1, wherein the sensor comprises a unitary sensor body.

3. The apparatus of claim 2, wherein the sensor body is made of plastics material.

4. The apparatus of claim 1, wherein the light source comprises a laser.

5. The apparatus of claim 1, wherein the photo-detector comprises a photo-transistor.

6. The apparatus of claim 1, wherein the sensor includes an aperture between the light source and the sample cell and an aperture between the sample cell and the photo-detector to define an illuminated viewing volume passing through the sample cell.

7. The apparatus of claim 6, wherein the apertures are substantially circular.

8. The apparatus of claim 6, wherein the viewing volume passes diametrically through the sample cell.

9. The apparatus of claim 6, wherein, where it strikes the outer surface of the sample cell, the beam of light is substantially perpendicular to the outer surface.

10. The apparatus of claim 1, wherein the signal processing means comprises a pulse height analyser.

11. The apparatus of claim 10, wherein the pulse height analyser is adapted to determine size/count distribution.

12. The apparatus of claim 1, wherein the signal processing means comprises a comparator.

13. The apparatus of claim 12, wherein the comparator is adapted to provide a number of pulses proportional to the number of particles detected in a predetermined time.

14. The apparatus of claim 13, wherein counting means is provided to count the number of pulses and to provide a signal representative of the result.
